(19) United States
(12) Patent Application Publication
(10) Pub. No.: US 2016/0257919 A1
(43) Pub. Date: Sep. 8, 2016

(54) DISPOSABLE BIOREACTOR

(71) Applicant: ZHENGZHOU VIRIGE BIOTECH CO., LTD., ZHENGZHOU (CN)

(72) Inventors: Zhiqiang Han, Zhengzhou (CN); Sheng Tong, Zhengzhou (CN); Huitao Liu, Zhengzhou (CN)

(73) Assignee: ZHENGZHOU VIRIGE BIOTECH CO., LTD.

(21) Appl. No.: 15/154,869

(22) Filed: May 13, 2016

Related U.S. Application Data
(63) Continuation of application No. PCT/CN2015/000132, filed on Mar. 5, 2015.

(30) Foreign Application Priority Data
Mar. 8, 2014 (CN) 201410082472.6

(51) Int. Cl. C12M 1/00; C12M 1/34

(52) U.S. Cl. CPC C12M 23/28; C12M 41/26; C12M 41/34

(57) ABSTRACT

The present invention belongs to the technical field of bioreactors, and particularly relates to a disposable bioreactor. The disposable bioreactor comprises a reactor cavity and a communicating pipe located at the top of the reactor cavity. The reactor cavity is formed by more than two cavity walls through fusion connection or adhesion connection, and is provided with an intersection part and at least one hollow arm part which is communicated with the communicating pipe through the intersection part in an intersection manner. The tail end of the hollow arm part is sealed. Liquid in the reactor cavity is driven by a rocking table, a plane convolution shaking table, and a three-dimensional convolution shaking table to flow along a long shaft of the disposable bioreactor, so that waves and turbulence are mixed. Compared with a method of performing eddy mixing and increasing gas-liquid exchange surface area in an existing reactor driven by a shaking table, the disposable bioreactor improves mixing and mass transfer efficiency greatly.

### CROSS-REFERENCE TO RELATED APPLICATIONS

This application is a continuation of International Patent Application No. PCT/CN2015/000132 with an international filing date of Mar. 5, 2015, designating the United States, now pending, and further claims priority benefits to Chinese Patent Application No. 201410082472.6, filed Mar. 8, 2014. The contents of all of the aforementioned applications, including any intervening amendments thereto, are incorporated herein by reference.

### TECHNICALFIELD

The present invention relates to the technical field of disposable bioreactors, and in particular to a disposable bioreactor.

### BACKGROUND

Non-intrusive in type bioreactors driven by shaking tables comprises a small culture tube, a spinner bottle, a conical flask, an inverted cone base bottle, a flat cone base bottle and a flat cone base tank as well as stirring type bioreactors of different sizes driven by stirring paddles. The non-intrusive mixing type bioreactors are bioreactors which are used for fermentation and cell suspension culture and applied most extensively at present. The non-intrusive mixing type bioreactors driven by shaking tables have the common characteristics that openings are located the ends of the bioreactors and are coaxial with long shafts of reactor cavities; except that the spinner bottle is placed horizontally, the openings are at the upper ends of the bioreactors and liquid in the bioreactors is driven in a rotation or convolution manner to generate eddy. The improvement of oxygen transfer of liquid by the generation of eddy is mainly by utilizing the principle of increasing liquid surface area, and the problem of low mixing efficiency remains in the manner of oxygen transfer by means of the increase of liquid surface area. Since the opening is located at one end of the long shaft of the reactor and is coaxial with the long shaft of the reactor cavity, in order to prevent liquid from splashing, it needs to increase the length of an upper gas channel, that is, an invalid channel for gas exchange through the reactor opening is enlarged, and thus the gas exchange efficiency is lowered. Though the stirring type bioreactor has high efficiency in mixing, only the mild stirring can be performed due to the limitation that shear force of the stirring paddles may lead to cellular damage and death, and this is also one of main defects of the stirring type bioreactor.

Moreover, the existing spinner bottles, conical flasks, inverted cone base bottles, flat cone base bottles and flat cone base tanks and stirring tank reactors of different sizes are made of hard materials such as glass, hard plastics and stainless steel. Since the reactors made of the glass and the stainless steel need to be reused, not only is the trouble in cleaning, disinfection and identification caused, but also there is the risk of cross contamination. Although the reactor formed by hard plastics through injection moulding is disposable, there is limitation in production process efficiency, used raw materials are relatively many, the reactor is fixed in shape and cannot undergo telescopic deformation, mutual nesting cannot be performed, the occupied space is large, and not only are the transport cost, irradiation cost and storage cost high, but also many wastes are generated to lead to more environmental problems. Though the disposable use of full hard micro reactors is feasible, for spinner bottles and reactors with larger sizes, particularly for reactors of a large production scale, the disposable use of these reactors formed by full hard materials through injection moulding will lead to great increase of production cost, irradiation cost, transport cost and storage cost and the generation of plenty of wastes, and seen from the angle of cost reduction and environmental protection, it is unfeasible. Therefore, it is a future bioreactor development trend to develop small reactors with process development and series amplification as the goal and large reactors with scale production as the goal and achieve disposable use, and a novel bioreactor which saves materials better, is higher in production efficiency, occupies less space and generates less environment waste needs to be further developed.

### SUMMARY

The objective of the present invention is to provide a disposable bioreactor, so as to solve the problems that an existing non-intrusive mixing type bioreactor driven by a shaking table is low in mixing and mass transfer efficiency.

In order to solve the aforementioned problem, the technical solution adopted by the present invention is as follows: a disposable bioreactor comprising a reactor cavity and a communicating pipe, wherein the communicating pipe is arranged at the top of the reactor cavity; and the reactor cavity is formed by more than two cavity walls through fusion connection or adhesion connection, and is provided with an intersection part and at least one hollow arm part which is communicated with the communicating pipe through the intersection part in an intersection manner. The tail end of the hollow arm part is sealed.

The hollow arm part is arranged horizontally.

The reactor cavity is provided with one hollow arm part, and the communicating pipe is located at the top of the reactor cavity and forms an L-shaped structure together with the hollow arm part.

The reactor cavity is provided with two hollow arm parts, and the communicating pipe is located at the top of the reactor cavity and forms an inversely T-shaped structure together with the hollow arm parts.

The reactor cavity is provided with two cavity walls, the two cavity walls are arranged up and down, wherein one cavity wall located on the upper portion is an upper cavity wall, and the other cavity wall is a lower cavity wall. At least one of the upper cavity wall and the lower cavity wall is a curved surface cavity wall.

One of the upper cavity wall and the lower cavity wall is a fixed-shaped supporting cavity wall capable of maintaining an inherent shape, and the other is a soft film cavity wall.

The fixed-shaped supporting cavity wall is formed by plastic raw materials through injection moulding or is formed by plastic sheets through plastics sucking moulding or blow molding.

The soft film cavity wall is formed by a plane plastic film or is formed by a deformable soft film bubble formed by the plane plastic film through plastics sucking moulding.

The upper cavity wall and the lower cavity wall are curved surface cavity walls. The fixed-shaped supporting cavity wall is an overlapped type cavity will capable of being overlapped in a sleeved manner, and the soft film cavity wall can be folded into the fixed-shaped supporting cavity wall so that the reactor cavity can be overlapped in a sleeved manner.

One of the upper cavity wall and the lower cavity wall is a curved surface cavity wall, and the other is a plane cavity wall.

The communicating pipe and the upper cavity wall are of an integrated structure.

An outer port of the communicating pipe is formed by punching after plastics sucking moulding, and a reinforcing ring which surrounds the outer port and remains after the punching is positioned at the position of the outer port of the communicating pipe.

The lower cavity wall is the curved surface cavity wall. The bioreactor further comprises a supporting fixing base which is provided with a yielding groove used for yielding to the lower cavity wall, and a fixing structure for fixing or pulling the reactor cavity is arranged on the periphery of a notch of the yielding groove.

The joint of the upper cavity wall and the lower cavity wall is provided with a joint flange which extends towards the periphery and is used for supporting, pulling and fixing the reactor cavity.

The reactor further comprises a cover for covering the upper cavity wall of the reactor cavity, and the cover is provided with a pipe sealing part for plugging a pipe opening of the communicating pipe.

The cover forms an overturning cover structure of the reactor cavity. One of two opposite sides of the cover is connected with the joint flange, and a fixed buckle is arranged between the other side of the cover and the joint flange.

The cavity wall of the reactor cavity is equipped with a pipe communicated with the reactor inner cavity so that liquid and gas can go in and out and dissolved oxygen, carbon dioxide and PH sensor electrodes can pass, and the pipe communicated with the reactor inner cavity and the connected cavity wall are of an integrally formed structure.

The reactor cavity is formed by two cavity walls through fusion connection or adhesion connection. One of the two cavity walls is a front cavity wall and the other is a rear cavity wall. The matching joint of the front cavity wall and the rear cavity wall is provided with a joint flange, and a hollow arm part of the reactor cavity is formed by corresponding parts of the front cavity wall and the rear cavity wall.

The disposable bioreactor has the beneficial effects that in the disposable bioreactor, the reactor cavity is provided with an intersection part and at least one hollow arm part which is communicated with the communicating pipe through the intersection part in an intersection manner, the tail end of the hollow arm part is sealed, and the communicating pipe is located at the top of the reactor cavity. Due to the existence of the hollow arm part, the disposable bioreactor can make liquid flow in a long and narrow space at least at the position of the hollow arm part. Since the communicating pipe is positioned at the top of the reactor cavity, the liquid flows along the long shaft of the reactor in the horizontal direction, the shaft of the communicating pipe for the reactor liquid to flow is in the vertical axial direction. When the disposable bioreactor is placed on a rocking table, a plane convolution shaking table or a three-dimensional convolution shaking table, the liquid can be made to flow in the hollow arm part of the reactor and does not splash. When reaching the sealed end of the hollow arm part, liquid flow writhes, returns and alternates up and down under the effect of turbulence, eddy and waves which are generated at the sealed end of the hollow arm part, and oxygen in the upper space is involved into the liquid flow in the process of writhing and alternating up and down; that is, liquid in the hollow arm part can writhe and alternate up and down continuously in the flowing process, so that the liquid can be in full contact with oxygen. Compared with a manner of mixing the liquid flow and oxygen by increasing the liquid flow surface area in the prior art, the mixing and mass transfer efficiency is improved greatly.

Furthermore, the curved surface cavity wall can achieve the effect of reducing reactor cavity wrinkles when pulling the reactor cavity. The fixed-shaped supporting cavity wall is more favorable for maintaining a three-dimensional space in the reactor cavity and is convenient to use. After the fixed-shaped supporting cavity wall is arranged to be an overlapped type cavity wall capable of being overlapped in a sleeved manner, the space occupied by the reactor can be further reduced, the transportation, irradiation and storage of the reactor are facilitated, and the cost is reduced. The reinforcing ring at the position of the outer port of the communicating pipe has the effects of improving communicating pipe opening strength and protecting a pipe cap from slippage. The supporting fixing base can help the fixation of the reactor cavity and facilitate large scale development of the reactor. The joint flange not only can have the effect of supporting the reactor cavity, but also provides a point of strength for pulling the reactor cavity. The cover can entirely cover and seal the upper cavity wall as well as the communicating pipe, the pipe sealing part of the cover can be used for plugging the pipe opening of the communicating pipe, the direct contact with the periphery of the communicating pipe opening is avoided when the cove is opened, and thus the pollution to the liquid is avoided. The overturning cover type cover can be prevented from being lost, and the buckle is used for fixing the cover conveniently. The cavity wall of the reactor cavity comprises pipes which are arranged on the soft film cavity walls and communicated with the reactor inner cavity so that liquid and gas can go in and out and dissolved oxygen, carbon dioxide and PH sensor electrodes can pass. The pipes are integrally thorned together with the reactor cavity wall, so the troublesome process of punching and fusion ring thermal synthesis welding between the pipes and the reactor cavity wall and particularly between the soft film cavity walls is omitted.

### BRIEF DESCRIPTION OF DRAWINGS

FIG. 1 is a structural schematic diagram of Embodiment 1 of a disposable bioreactor;

FIG. 2 is an exploded view of Embodiment 1 of a disposable bioreactor;

FIG. 3 is a first working principle diagram of Embodiment 1 of a disposable bioreactor;

FIG. 4 is a second working principle diagram of Embodiment 1 of a disposable bioreactor;

FIG. 5 is a matching schematic diagram of a communicating pipe and a pipe cap in FIG. 1;

FIG. 6 is a structural schematic diagram of Embodiment 2 of a disposable bioreactor;

FIG. 7 is a structural schematic diagram of Embodiment 3 of a disposable bioreactor;

FIG. 8 is an exploded view of Embodiment 3 of a disposable bioreactor;

FIG. 9 is a structural schematic diagram of Embodiment 4 of a disposable bioreactor;

FIG. 10 is a structural schematic diagram of Embodiment 5 of a disposable bioreactor;

FIG. 11 is an exploded view of Embodiment 5 of a disposable bioreactor;

FIG. 12 is a side-by-side placing schematic diagram of Embodiment 5 of as disposable bioreactor;

FIG. 13 is a structural schematic diagram of Embodiment 6 of a disposable bioreactor;

FIG. 14 is a structural schematic diagram of Embodiment 7 of a disposable bioreactor;

FIG. 15 is a structural schematic diagram of Embodiment 7 of a disposable bioreactor after liquid is contained;

FIG. 16 is a sleeved overlapping schematic diagram of Embodiment 7 of a disposable bioreactor;

FIG. 17 is a structural schematic diagram of Embodiment 8 of a disposable bioreactor;

FIG. 18 is a structural schematic diagram of Embodiment 8 of a disposable bioreactor after liquid is contained;

FIG. 19 is a sleeved overlapping schematic diagram of Embodiment 8 of a disposable bioreactor;

FIG. 20 is a structural schematic diagram of Embodiment 9 of a disposable bioreactor;

FIG. 21 is a structural schematic diagram of Embodiment 9 of a disposable bioreactor after liquid is contained;

FIG. 22 is a sleeved overlapping schematic diagram of Embodiment 9 of a disposable bioreactor;

FIG. 23 is a structural schematic diagram of Embodiment 10 of a disposable bioreactor;

FIG. 24 is a structural schematic diagram of Embodiment 10 of a disposable bioreactor after liquid is contained;

FIG. 25 is an overlapping schematic diagram of Embodiment 10 of a disposable bioreactor;

FIG. 26 is a structural schematic diagram of Embodiment 11 of a disposable bioreactor;

FIG. 27 is a structural schematic diagram of Embodiment 12 of a disposable bioreactor;

FIG. 28 is a structural schematic diagram of Embodiment 13 of a disposable bioreactor;

FIG. 29 is a structural schematic diagram of Embodiment 14 of a disposable bioreactor;

FIG. 30 is a structural schematic diagram of Embodiment 15 of a disposable bioreactor;

FIG. 31 is a structural schematic diagram of Embodiment 16 of a disposable bioreactor;

FIG. 32 is a structural schematic diagram of Embodiment 17 of a disposable bioreactor;

FIG. 33 is a structural schematic diagram of Embodiment 18 of a disposable bioreactor;

FIG. 34 is a structural schematic diagram of Embodiment 19 of a disposable bioreactor;

FIG. 35 is a structural schematic diagram of Embodiment 20 of a disposable bioreactor;

FIG. 36 is a structural schematic diagram of Embodiment 21 of a disposable bioreactor; and

FIG. 37 is a structural schematic diagram of Embodiment 22 of a disposable bioreactor.

### DESCRIPTION OF EMBODIMENTS

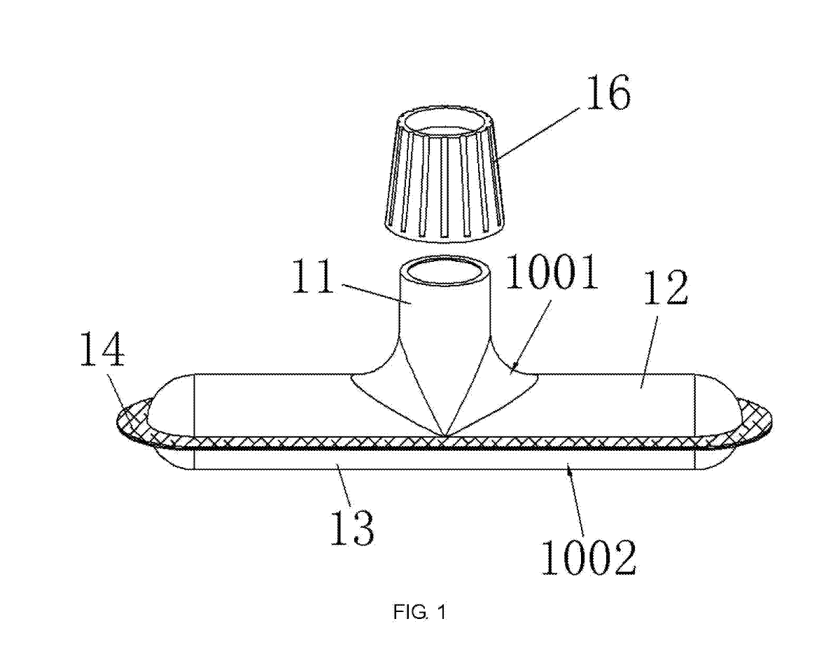

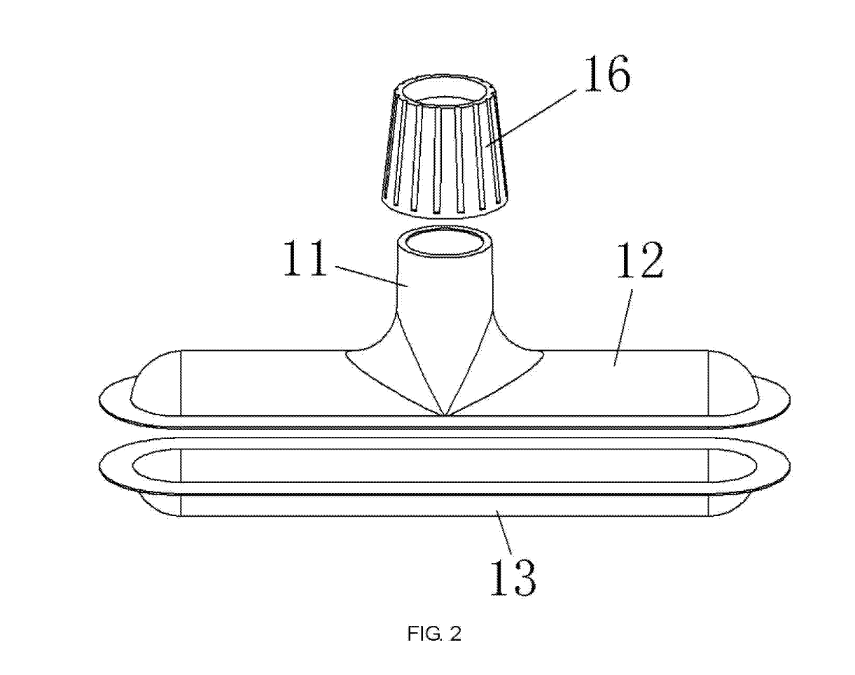

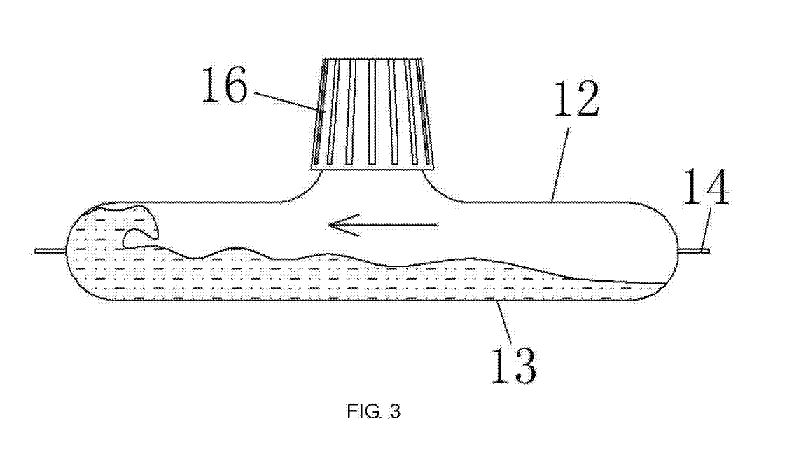

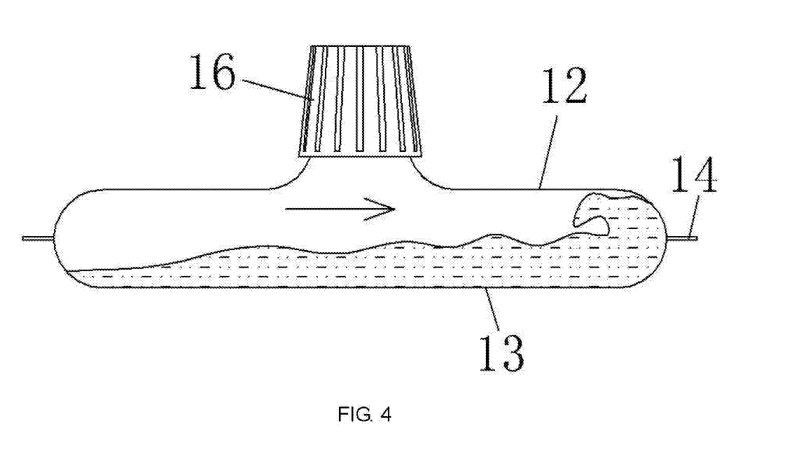

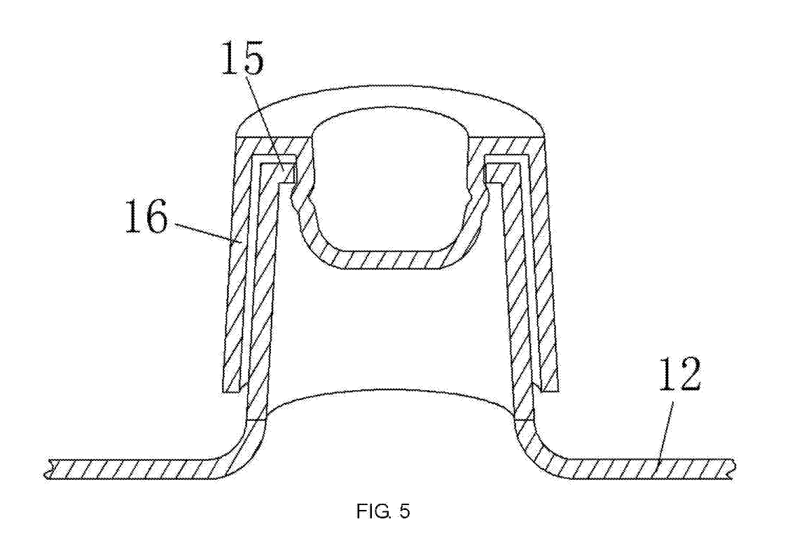

Embodiment 1 of a disposable bioreactor is as shown in FIG. 1-5. The disposable bioreactor comprises a reactor cavity and a communicating pipe 11. The reactor cavity is made of a non-toxic and harmless plastic material, and the requirements of disposable use can be met. As can be seen from FIGS. 1 and 2, the reactor cavity is formed by fusing two cavity walls. The two cavity walls are arranged up and down, wherein one cavity wall located on the upper portion is an upper cavity wall 12, and the other cavity wall located on the lower portion is a lower cavity wall 13. A joint flange 14 is formed at the fused position of the upper cavity wall and the lower cavity wall. In case of need, the reactor cavity can be supported and pulled through the joint flange 14. The communicating pipe 11 is located on the upper cavity wall 12, and the communicating pipe 11 and the upper cavity wall 12 are of an integrated structure. In this embodiment, the upper cavity wall and the lower cavity wall are fixed-shaped supporting cavity walls, namely cavity walls capable of maintaining inherent shapes thereof. The upper cavity wall and the lower cavity wall are of a bubble shell structure formed by plastic sheets through plastics sucking moulding (in other embodiments, the upper cavity wall and the lower cavity wall can be fixed-shaped supporting cavity walls formed by plastic sheet materials through blow molding or plastic raw materials through injection moulding), wherein the concrete plastic sheet materials can be raw materials or sheets or coiled materials such as polypropylene (PP), polymethylene (PE), polyethylene glycol terephthalate (PET) (comprising amorphous polyethylene terephthalate (APET) and polyethylene terephthalate glycol (PETG)), polyvinyl chloride (PVC), ABS, PC, PS GAG and acrylic which are formed through injection moulding or plastics sucking moulding, as well as compound plastics sucking moulding plastic sheets and coiled materials, wherein the compound plastics sucking moulding coiled materials comprise PVC, PET, PP, PS and thermoplastic coiled materials such as PVC/PE, PET/PE and HIPS. Moreover, as can be seen from FIG. 2, the upper cavity wall and the lower cavity wall are curved surface cavity walls, that is, the upper cavity wall and the lower cavity wall are non-planar cavity walls, so that the upper cavity wall and the lower cavity wall are fused, a three-dimensional space is naturally formed between them.

On the whole, the reactor cavity in this embodiment comprises an intersection part 1001 and two hollow arm parts 1002. The tail ends of the hollow arm parts 1002 are sealed, and the blind ends of the hollow arm parts 1002 are hemispherical. Due to the arrangement of the hollow arm parts 1002, a thin and long channel is formed in the reactor cavity, and thus liquid can perform reciprocating flow in the reactor cavity along the long shaft of the reactor (namely in the extension direction of the corresponding hollow arm parts). In this embodiment, the cross sections of the hollow arm parts 1002 are round (in other embodiments, the cross sections of the hollow arm parts may further be oval, polygonal, rectangular, triangular, semicircular, semioval, trapezoidal or in different combined shapes of the same inner cavity formed by the combination of the aforementioned geometrical figures), and the hollow arm parts are formed by splicing corresponding parts of the upper cavity wall and the lower cavity wall. Due to the arrangement of the hollow arm parts 1002, the length-diameter ratio of the reactor cavity in the extending direction of the hollow arm parts 1002 is larger than or equal to 1.5. The center lines of the two hollow arm parts 1002 are collinear, and at the position of the intersection part 1001 of the cavity, the two hollow arm parts 1002 are communicated with the communicating pipe 11 through the intersection part 1001 of the cavity, and thus the communicating pipe 11 and the hollow arm parts 1002 of the reactor cavity together form an inversely T-shaped structure.

It has been mentioned above that the communicating pipe 11 and the upper cavity wall 12 are of an integrated structure, the upper cavity wall 12 is formed by plastics sucking moulding, and thus the communicating pipe 11 is also formed by plastics sucking moulding. After the plastics sucking moulding, the outer end of the communicating pipe 11 is non-transparent preliminarily, and a bard shell structure with a certain thickness is formed at the position of the outer end of the communicating pipe 11. The outer port of the communicating pipe 11 is formed on the hard shell structure by punching. After the punching, a reinforcing ring 15 is formed at the position of the outer port of the communicating pipe by the hard shell structure. The reinforcing ring 15 can be directly used for being clamped and matched with a pipe cap 16 (a cover) of the communicating pipe 11, and the communicating pipe is a common channel for reaction mass and gas exchange.

When the disposable bioreactor is placed on a rocking table, a plane convolution shaking table or a three-dimensional convolution shaking table, the liquid can be made to flow in the reactor cavity along the long shaft, and through improvement of potential energy and liquid kinetic energy at one end, the liquid is driven to flow towards the low end of the potential energy and liquid kinetic energy, and wave mixing and turbulent mixing are performed; and at the blind end of the hollow arm part, the arc-shaped tangent of a pipe wall part forms turbulent mixing. When the rocking table is used, the center lines of the two hollow arm parts are kept consistent with the rocking direction. When the disposable bioreactor is slantly placed on a plane convolution motion platform to perform convolution motion, actually the liquid is tossed upwards along an inclined plane and then the gravity of the liquid slips down along the tangent, so that the wave type or two-end turbulence type mixing and gas exchange inside the hollow arm part are formed.

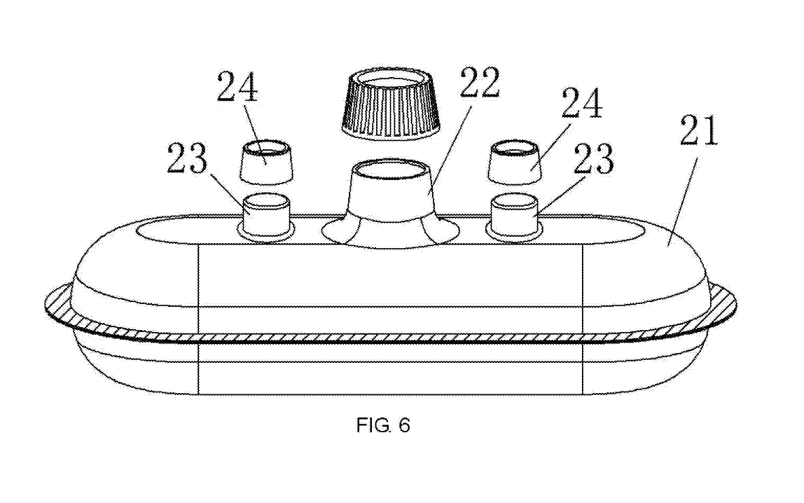

Embodiment 2 of the disposable bioreactor is as shown in FIG. 6. This embodiment is different from Embodiment 1 in that in this embodiment, an upper cavity wall 21 is of a flat top structure. Besides a communicating pipe 22, the top of the upper cavity wall 21 is further provided with a gas exchange pipe 23. The gas exchange pipe 23 and the upper cavity wall 21 are also of an integrated structure, and the outer end of the gas exchange pipe 23 is provided with a gas exchange pipe cap 24.

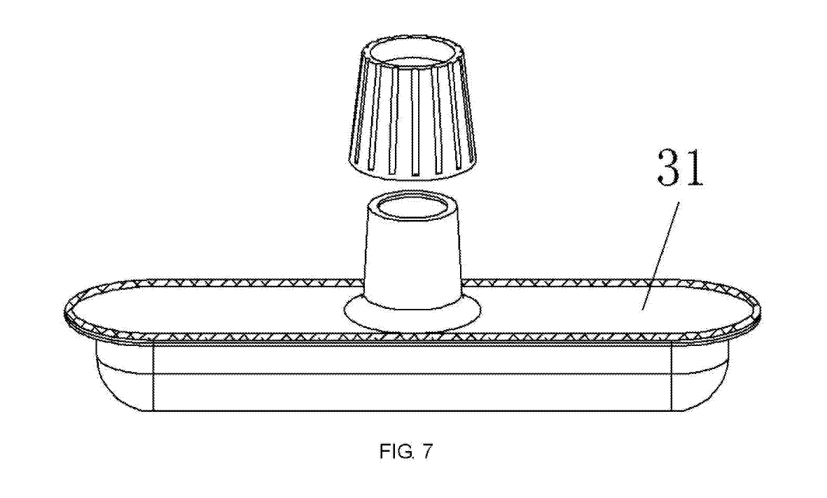

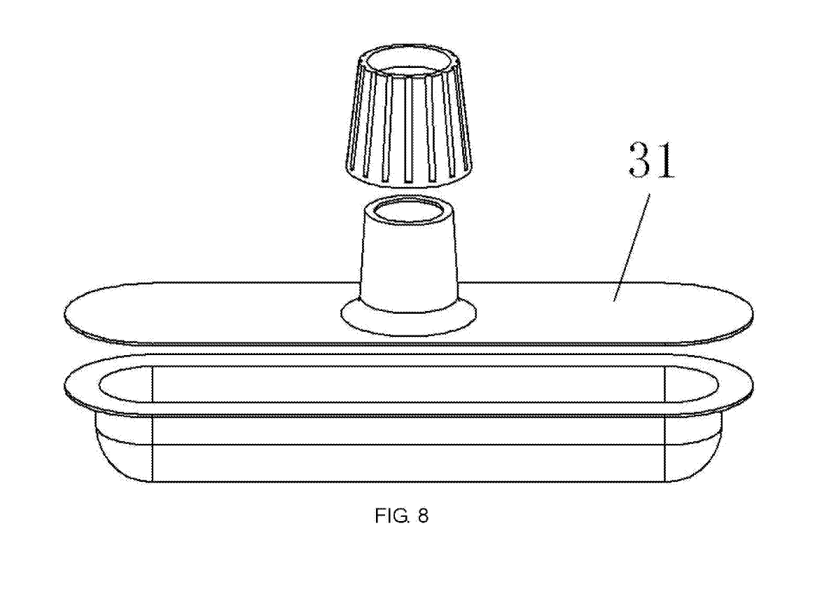

Embodiment 3 of the disposable bioreactor is as shown in FIGS. 7-8. This embodiment is different from Embodiment 1 in that in this embodiment, an upper cavity wall 31 of the reactor cavity is a plane cavity wall.

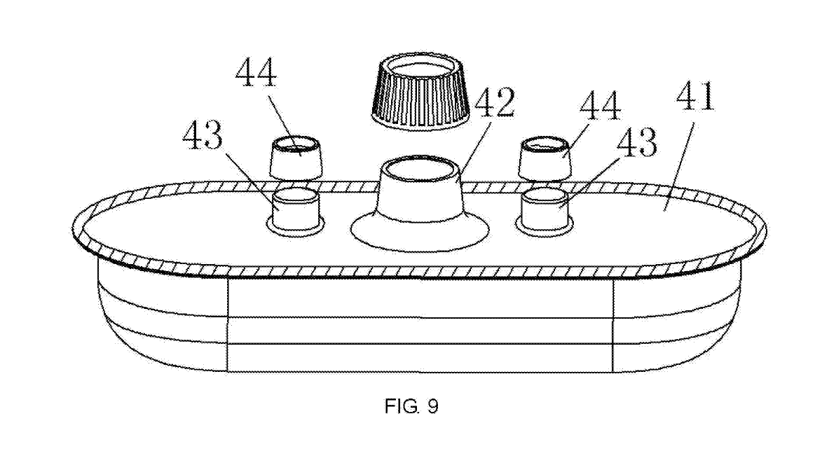

Embodiment 4 of the disposable bioreactor is as shown in FIG. 9. This embodiment is different from Embodiment 3 in that in this embodiment, an upper cavity wall 41 is of a flat to structure. Besides a communicating pipe 42, the top of the upper cavity wall 41 is further provided with a gas exchange pipe 43. The gas exchange pipe 43 and the upper cavity wall 41 are also of an integrated structure, and the outer end of the gas exchange pipe 43 is provided with a gas exchange pipe cap 44.

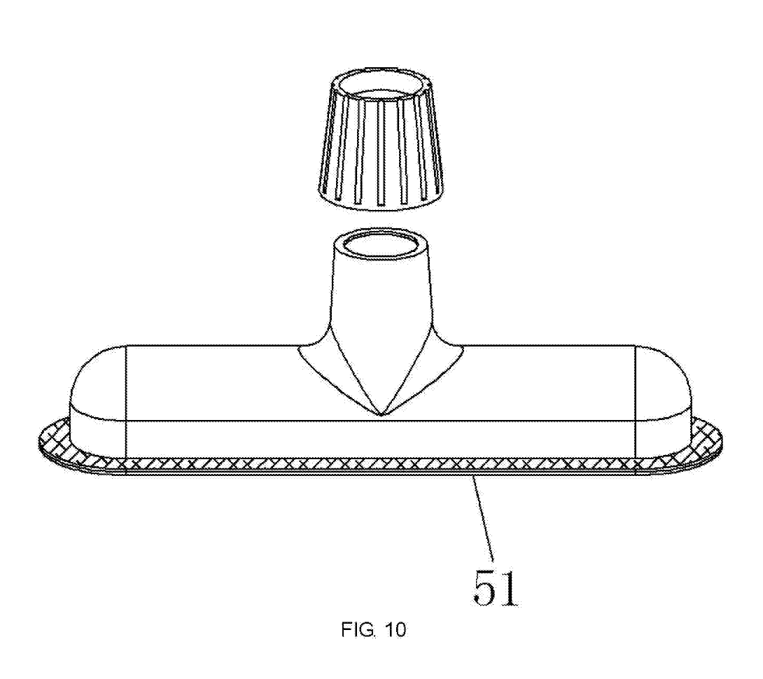

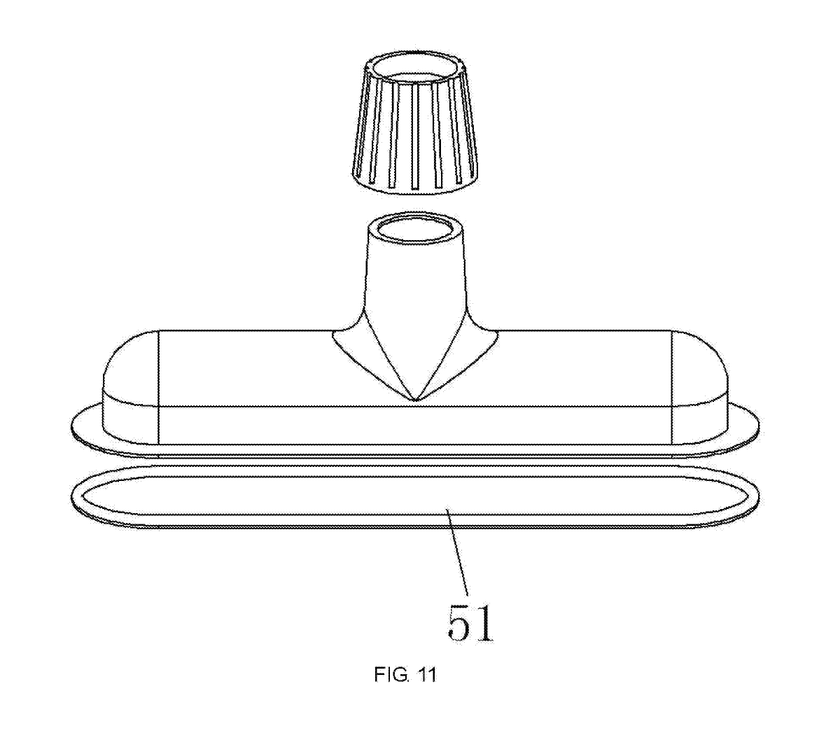

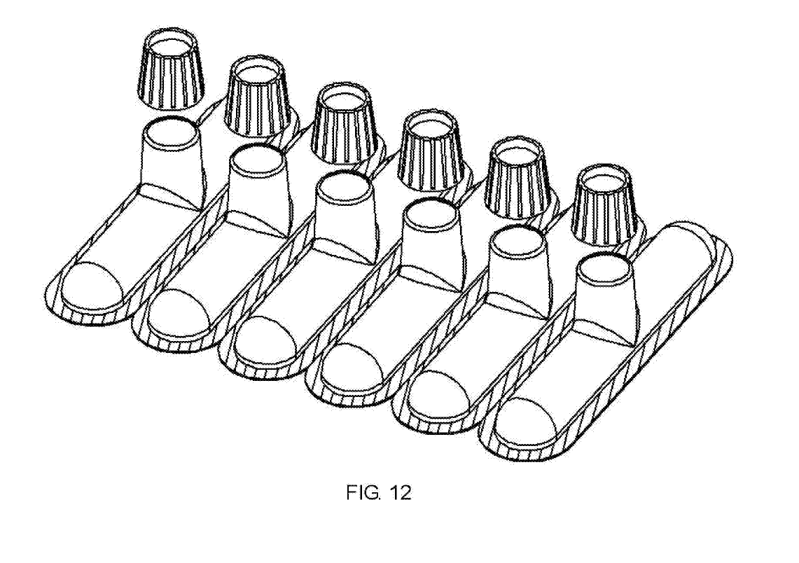

Embodiment 5 of the disposable bioreactor is as shown in FIGS. 10-12. This embodiment is different from Embodiment 1 in that in this embodiment, a lower cavity wall 5 of the reactor cavity is a plane cavity wall, and after the lower cavity wall with the structure is adopted, the reactor cavity can serve as a small reactor pipe free of a pipe frame to maintain a standing position automatically, and is convenient to use.

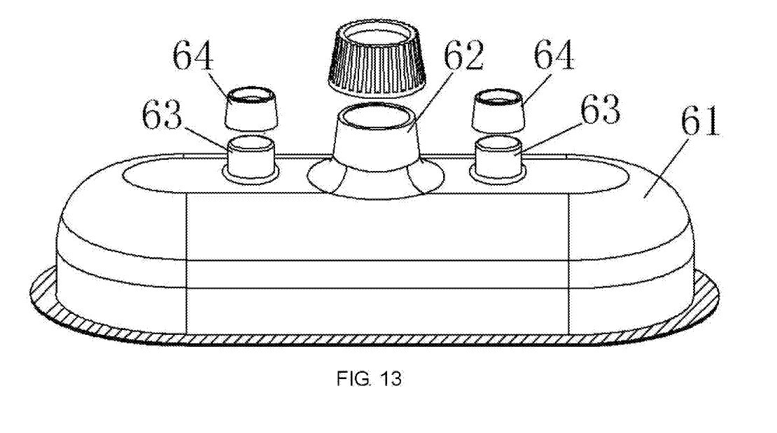

Embodiment 6 of the disposable bioreactor is as shown in FIG. 13. This embodiment is different from Embodiment 5 in that in this embodiment, an upper cavity wall 61 is of a flat top bubble shell structure, and besides a communicating pipe 62, the top of the upper cavity wall 61 is further provided with a gas exchange pipe 63. The gas exchange pipe 63 and the upper cavity wall 61 are also of an integrated structure, and the outer end of the gas exchange pipe 63 is provided with a gas exchange pipe cap 64.

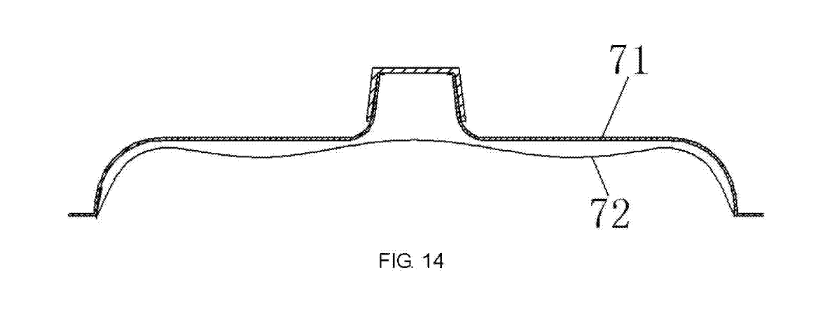

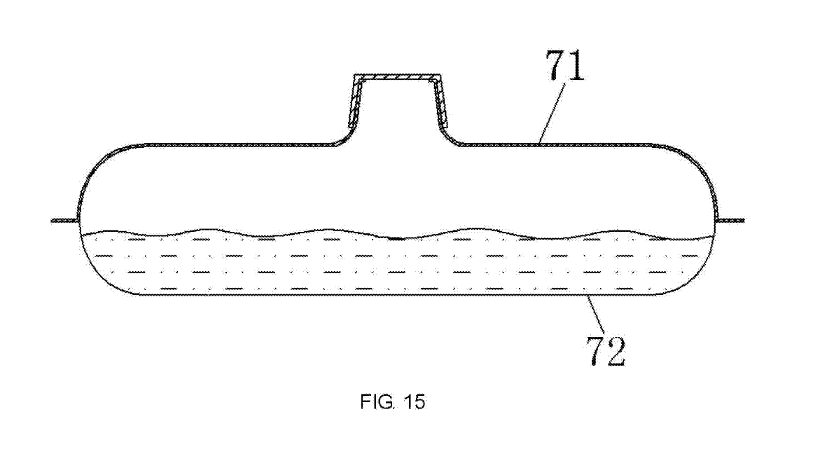

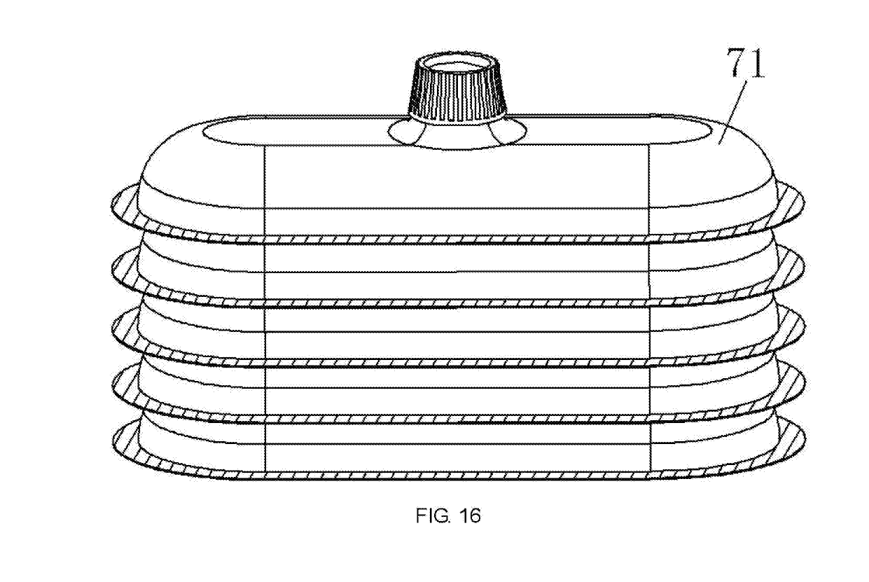

Embodiment 7 of the disposable bioreactor is as shown in FIGS. 14-16. This embodiment is different from Embodiment 1 in that in this embodiment, an upper cavity wall 71 of the reactor cavity is a sleeved overlapped type cavity wall with the small top and large bottom. A lower cavity wall 72 is a soft film cavity wall, the shape of the lower cavity wall 72 (except the structure without the communicating pipe) is adaptive with the shape of the upper cavity wall 71, and the lower cavity wall is concretely formed by a soft film bubble formed by plastics sucking moulding, the soft film bubble is a flexible film with a curved surface structure formed by a plane soft film through plastics sucking drawing moulding. The plane soft film can be a polyethylene (PE) film, a low-density polyethylene (LDPE) film, a linear low density polyethylene (LLDPE) film, a polypropylene (PP) film, a polyvinyl chloride (PVC) film, a polyethylene glycol terephthalate (PET) film, a polystyrene (PS) film, an ethylene/acetic acid (EVA) film, a polyamide (PA) film, a multilayer composite film of the combination of the aforementioned different resin films, a multilayer coextrusion film formed by multilayer coextrusion of the aforementioned resins, and multilayer coextrusion films such as a PP/SEBS/PP coextrusion film containing SEBS and EVOH, a PA/EVOH/PE coextrusion film and PA/EVOH/PP. In the transport, irradiation and storage process, the lower cavity wall 72 is overturned upwards to be embedded into the upper cavity wall 71, so that the reactor cavities can be mutually sleeved and overlapped (as shown in FIG. 16), and the transport cost, irradiation cost and storage cost of the reactor are reduced. In the using process, because the upper cavity wall 71 is a fixed-shaped supporting cavity wall, the upper cavity wall 71 can achieve the effect of supporting the reactor cavity. After the liquid is filled, under the effect of droop gravity of liquid, the lower cavity wall 72 can automatically move out of the upper cavity wall 71 and automatically maintain a three-dimensional shape under the effect of liquid gravity (as shown in FIG. 15).

Since the soft film is characterized by being elastic and low in shear force, one side is designed into a rigid supporting bubble shell structure, and the other side is provided with the soft film. Therefore, the rigid supporting bubble shell structure characteristics of simplicity in structure and convenience in operation are utilized, the shortcoming that only by means of an air blowing system can a full soft film reactor maintain a three-dimensional internal space is overcome, and the soft film advantage of buffering shear force is utilized to buffer the shear damage to cells caused by liquid flow impact. By utilizing the deformation characteristics of the soft film and the sleeving and overlapping advantages of a hard bubble shell, in use, the soft film bubble matched in shape is embedded into the hard bubble shell so as to save space. In fact, considered from the economic aspect and energy saving and environmental protection aspect, it is unacceptable that the large full hard reactor is disposable. However, the soft and hard embedding reactor according to the invention not only reduces production cost, transport cost, irradiation cost and storage cost, but also reduces generated wastes, and thus the reactor is disposable.

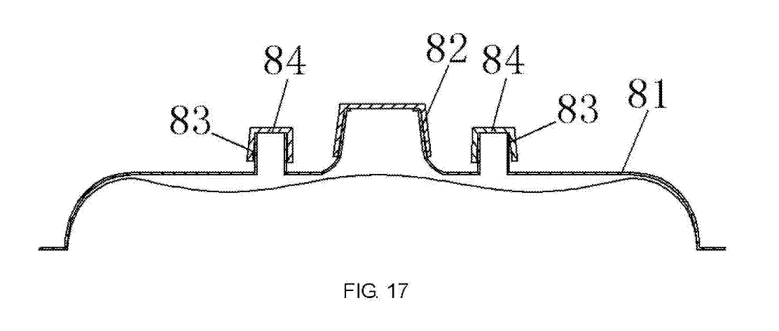

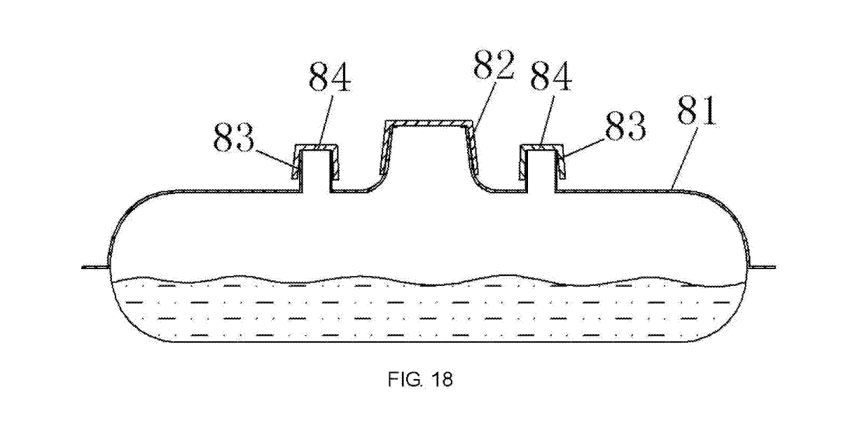

Embodiment 8 of the disposable bioreactor is as shown in FIGS. 17-19. This embodiment is different from Embodiment 7 in that in this embodiment, besides a communicating pipe 82, the top of an upper cavity wall 81 is further provided with a has exchange pipe 83. The gas exchange pipe 83 and the upper cavity wall 81 are also of an integrated structure, and the outer end of the gas exchange pipe 83 is provided with a gas exchange pipe cap 84.

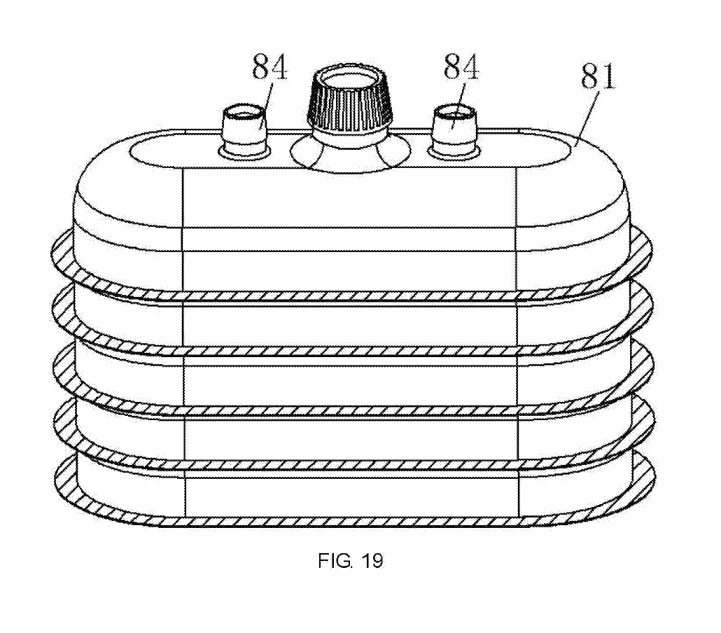

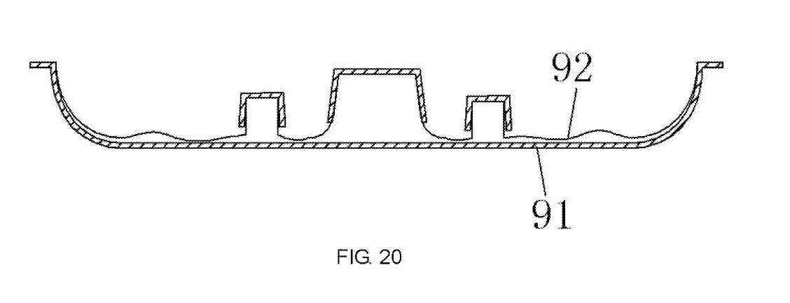

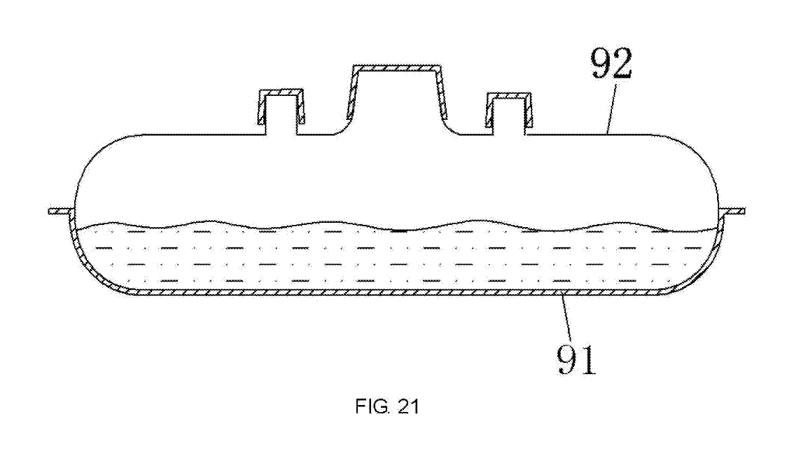

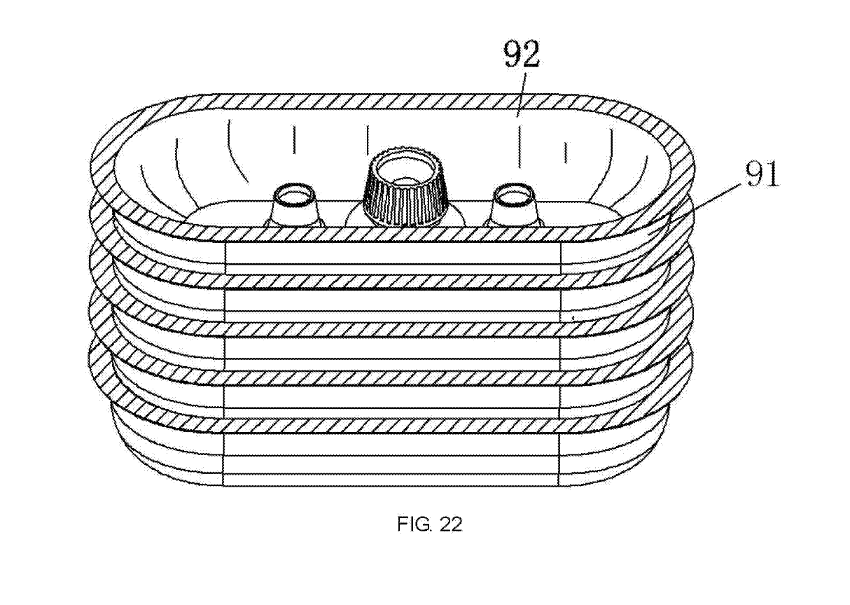

Embodiment 9 of the disposable bioreactor is as shown in FIGS. 20-22. This embodiment is different from Embodiment 2 in that in this embodiment, a lower cavity wall 91 of the reactor cavity is a sleeved overlapped type cavity all with the large top and the small bottom. An upper cavity wall 92 is a soft film cavity wall. Except that there is no communicating pipe, air inlet pipe or air outlet pipe, the shape of the upper cavity wail 92 is adaptive with the shape of the lower cavity wall 91, so in the transport and irradiation process, the upper cavity wall 92 can be folded into the lower cavity wall 91, the reactor cavity is overlapped in a sleeve manner (as shown in FIG. 19), and the transport cost and irradiation cost of the reactor are reduced.

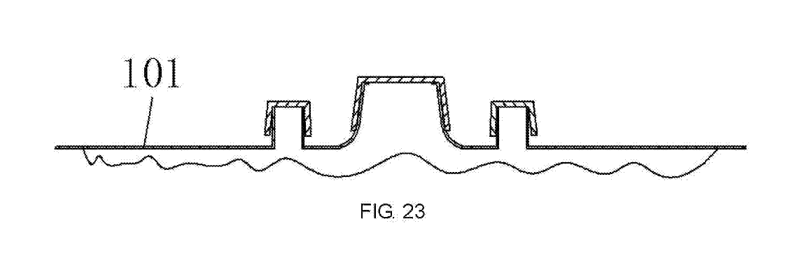

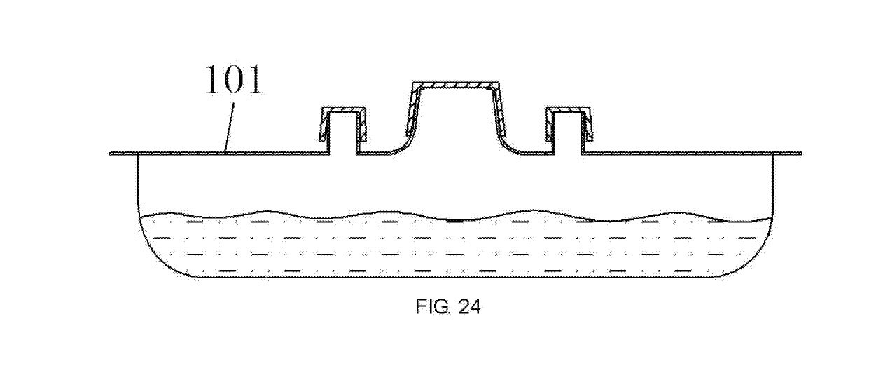

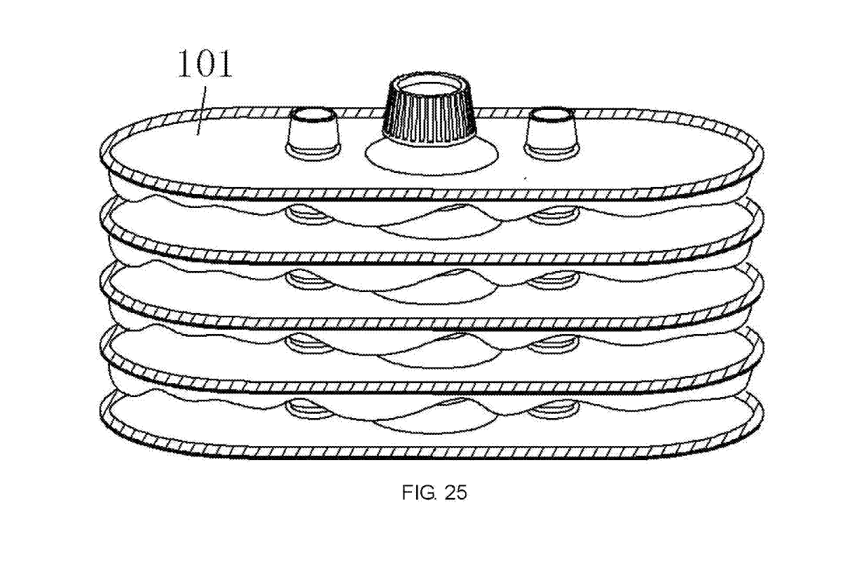

Embodiment 10 of the disposable bioreactor is as shown in FIGS. 23-25. This embodiment is different from Embodiment 8 in that in this embodiment, upper cavity wall 101 of the reactor cavity is a plane cavity wall.

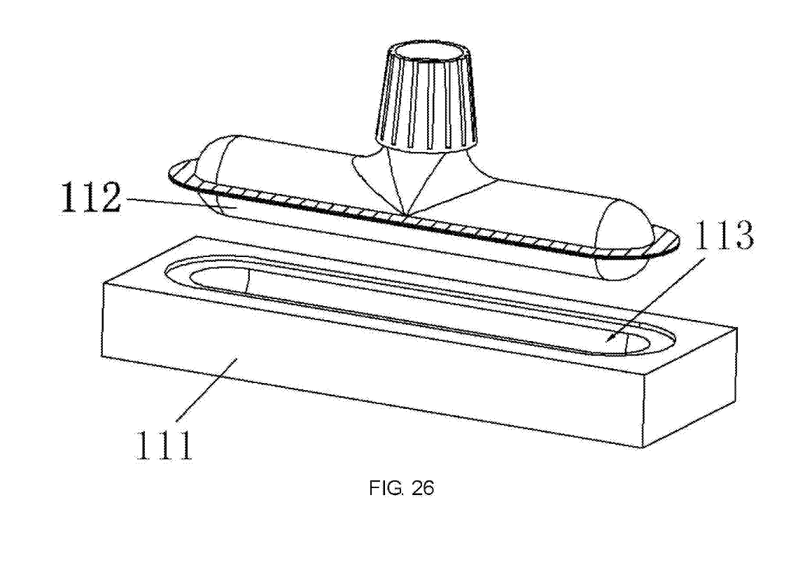

Embodiment 11 of the disposable bioreactor is as shown in FIG. 26. This embodiment is different from Embodiment 1 in that in this embodiment, the disposable bioreactor further comprises a supporting fixing base 111, and the supporting fixing base 111 is provided with a yielding groove 113 for yielding to the lower cavity wall 112.

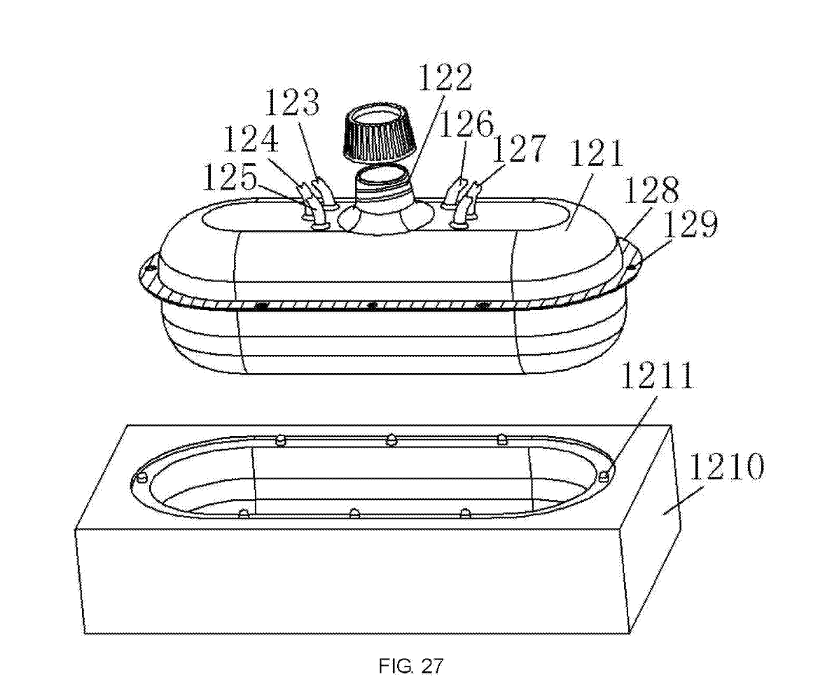

Embodiment 12 of the disposable bioreactor is as shown in FIG. 27. This embodiment is different from Embodiment 11 in that in this embodiment, an upper cavity wall 121 is a fiat top bubble shell structure, and besides a communicating pipe 122, the top of the upper cavity wall 121 is further provided with a gas exchange pipe 123, a liquid inlet pipe 124, a liquid taking pipe 125, an oxygen and carbon dioxide sensor electrode sleeve 126 and a PH sensor electrode sleeve 127. The gas exchange pipe 123 and the upper cavity wall 121 are also of an integrated structure, and the outer end of the gas exchange pipe 123 is provided with a gas bacterial filter (not shown in the figure). The gas exchange pipe, the liquid inlet pipe, the liquid taking pipe, the oxygen and carbon dioxide sensor electrode sleeve and the PH sensor electrode sleeve are also integrally formed together with the upper cavity wall 121 through plastics sucking moulding. Moreover, a joint flange 128 of the reactor cavity is provided with a pulling fixing hole 129, and a fixing supporting base 1210 is provided with a ball head 1211 corresponding to the pulling fixing hole. In use, the fixing supporting base 1210 can be matched with the pulling fixing hole 129 in the joint flange 128 through the ball head 1211 so as to fix the reactor cavity.

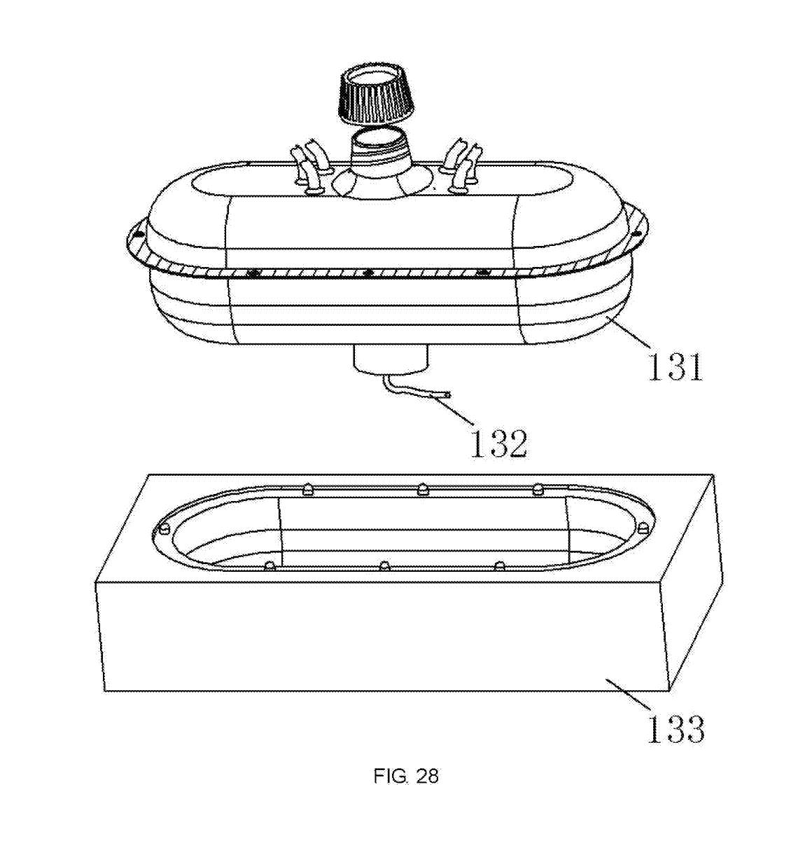

Embodiment 13 of the disposable bioreactor is as shown in FIG. 28. This embodiment is different from Embodiment 11 in that in this embodiment, the bottom of a lower cavity wall 131 is further provided with a bottom air inlet pipe 132; moreover, a heating component (not shown in the figure) is further arranged in a groove of a fixing supporting base 133, and the heating component is concretely a heating device with the temperature regulated automatically, such as an electric blanket or a heat exchanger

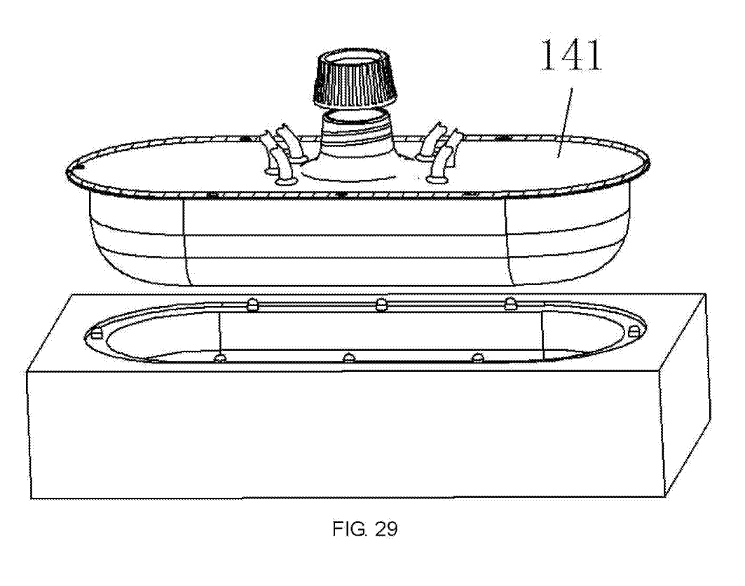

Embodiment 14 of the disposable bioreactor is as shown in FIG. 29. This embodiment is different from Embodiment 12 in that in this embodiment, an upper cavity wall 141 of the reactor cavity is a plane cavity wall; moreover, in this embodiment, the upper cavity wall 141 is a fixed-shaped supporting cavity wall. In this embodiment, the upper cavity wall 141 can further be a plane soft film cavity wall, and the plane soft film material can be selected by referring to Embodiment 7.

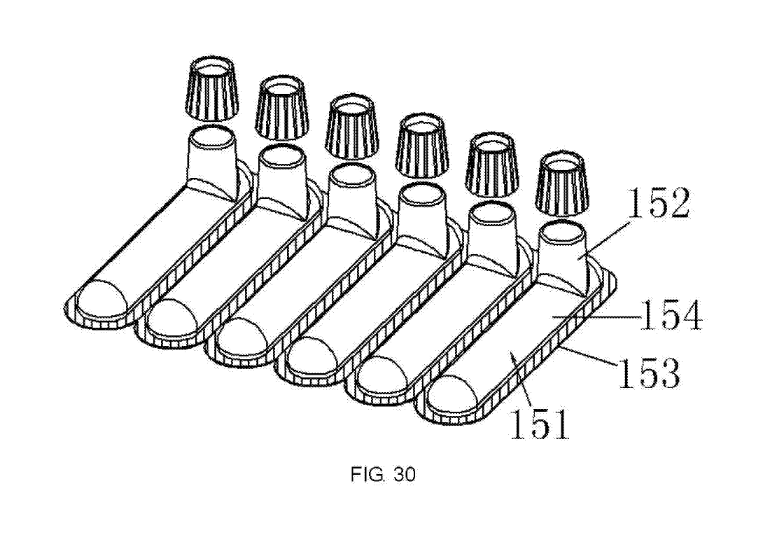

Embodiment 15 of the disposable bioreactor is as shown in FIG. 30. This embodiment is different from Embodiment 1 in that in this embodiment, the reactor cavity is only provided with a hollow arm part 151, and thus a communicating pipe 152 and the hollow arm part 151 in the reactor cavity form an L-shaped structure. In addition, in this embodiment, a lower cavity wall 153 of the reactor cavity is a plane cavity wall. FIG. 30 shows the situation that six reactors are placed in parallel. During production, a plurality of upper cavity walls 154 are formed on a material by one-time plastics sucking moulding or injection moulding, and then the upper cavity walls 154 adhere to or are fused with a sheet so that a plurality of reactor cavities can be obtained in one time.

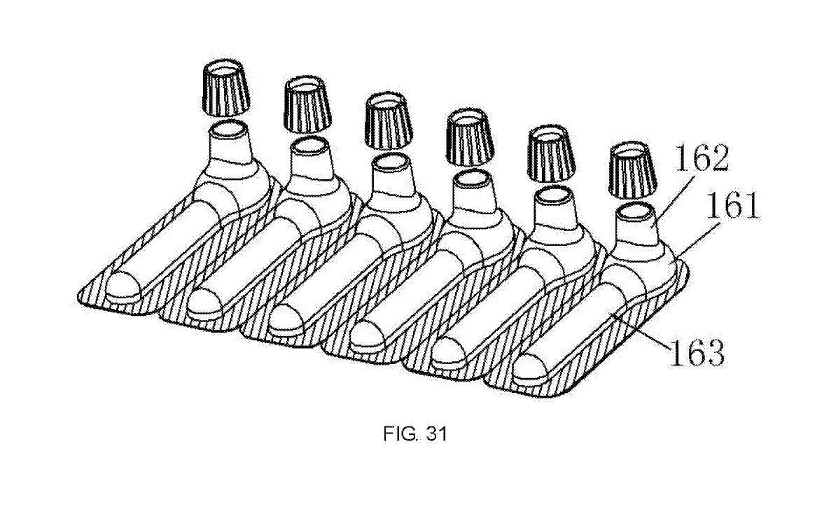

Embodiment 16 of the disposable bioreactor is as shown in FIG. 31. This embodiment is different from Embodiment 15 in that in this embodiment, an intersection part 161 of the reactor cavity forms a ballooning structure, and the intersection part 161 with the ballooning structure can further facilitate the communication between a communicating pipe 162 and a hollow arm part 163 of the reactor cavity and prevent too much liquid in the hollow arm part 163 from overflowing into the communicating pipe 162.

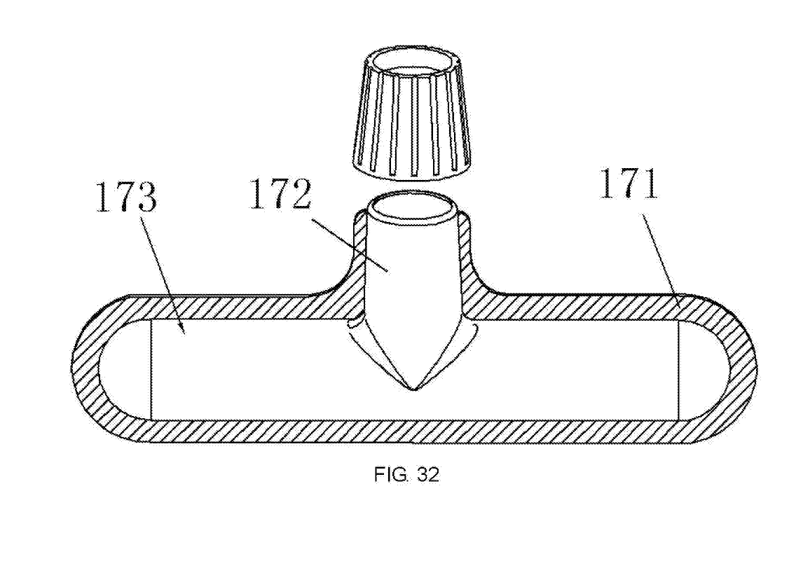

Embodiment 17 of the disposable bioreactor is as shown in FIG. 32. This embodiment is different from Embodiment 1 in that in this embodiment, the two cavity walls of the reactor cavity are arranged in a front-and-rear manner, the cavity wall located in the front is a front cavity wall, and the other is a rear cavity wall. The front cavity wall and the rear cavity wall are connected through fusion, and a joint flange 171 is formed at the fusion position of the front cavity wall and the rear cavity wall. Moreover, as can be seen from FIG. 32, in this embodiment, a communicating pipe 172 and a hollow arm part 173 of the reactor cavity are formed by splicing the front cavity wall and the rear cavity wall.

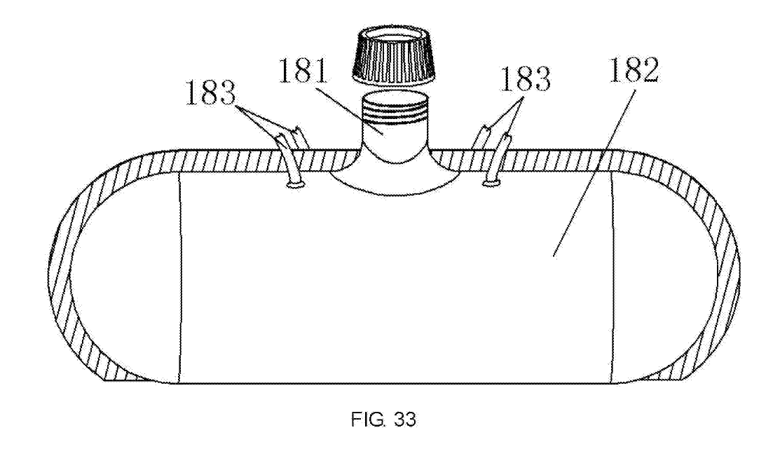

Embodiment 18 of the disposable bioreactor is as shown in FIG. 33. This embodiment is different from Embodiment 17 in that in this embodiment, a communicating pipe 181 is located on a front cavity wall 182; moreover, the front cavity wall and a rear cavity wall are further respectively provided with communicating pipes 183 which are connected with external corresponding components (a liquid inlet pipe and O2, CO2 and PH sensors and the like).

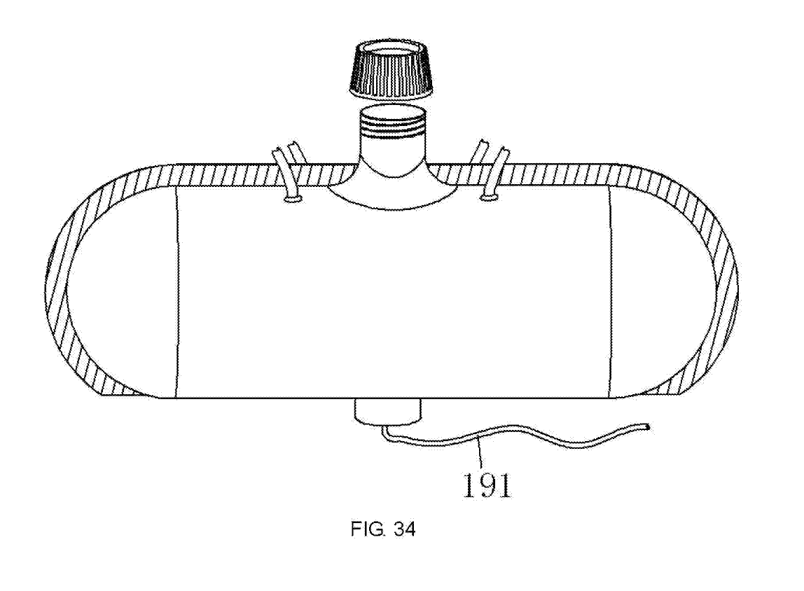

Embodiment 19 of the disposable bioreactor is as shown in FIG. 34. This embodiment is different from Embodiment 18 in that in this embodiment, the bottom of the reactor cavity is further provided with an air inlet pipe 191.

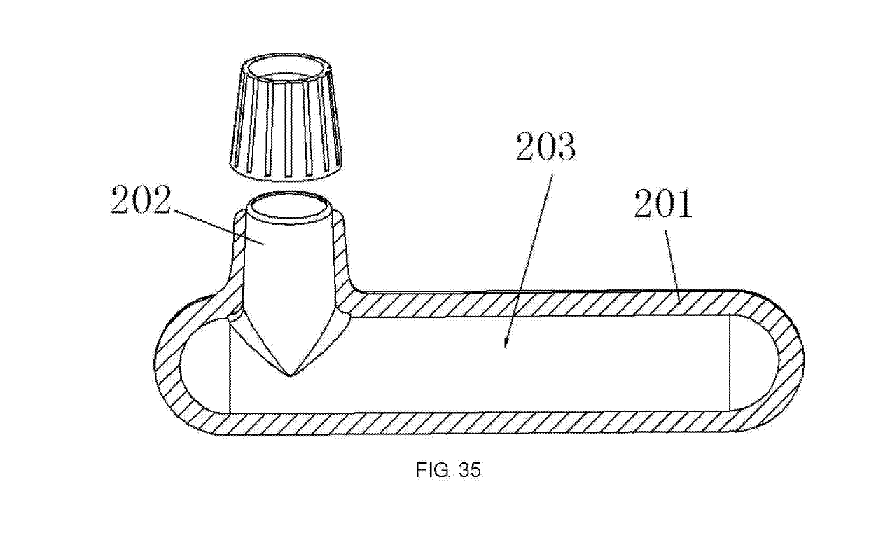

Embodiment 20 of the disposable bioreactor is as shown in FIG. 35. This embodiment is different from Embodiment 15 in that in this embodiment, the two cavity walls of the reactor cavity are arranged in a front-and-rear manner, the cavity wall located in the front is a front cavity wall, and the other is a rear cavity wall. The front cavity wall and the rear cavity wall are connected through fusion, and a joint flange 201 is formed at the fusion position of the front cavity wall and the rear cavity wall. Moreover, as can be seen from FIG. 32, in this embodiment, a communicating pipe 202 and a hollow arm part 203 of the reactor cavity are formed by splicing the front cavity wall and the rear cavity wall.

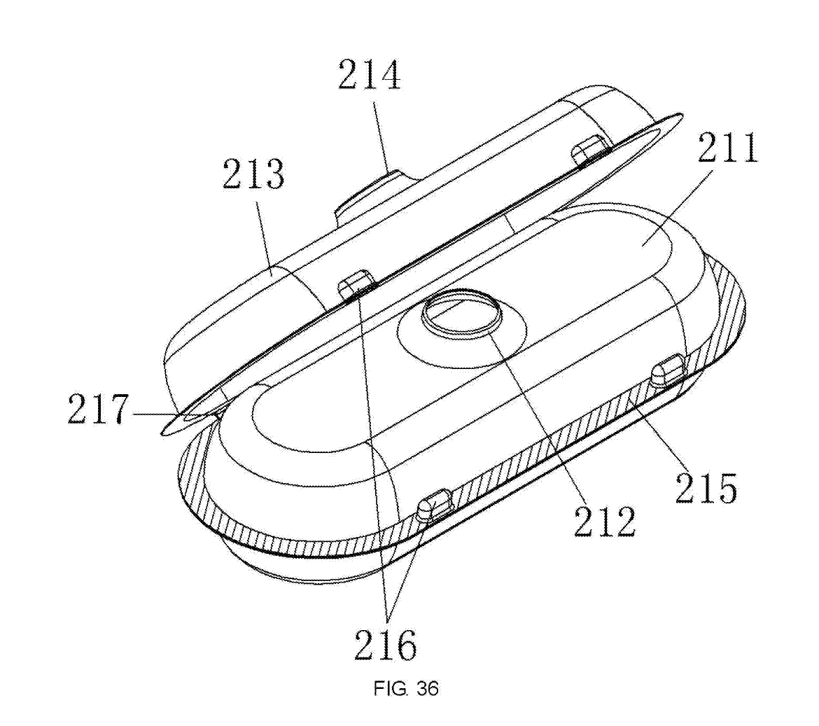

Embodiment 21 of the disposable bioreactor is as shown in FIG. 36. This embodiment is different from Embodiment 2 in that in this embodiment, a gas exchange pipe, a gas exchange pipe cap and a pipe cap of a communicating pipe 212 are omitted on an upper cavity wall 211 of the reactor cavity. Moreover, the reactor in this embodiment is further provided with a cover 213, and the cover 213 is in fact an extension structure of the pipe cap of the communicating pipe 212 and is used for entirely covering the upper cavity wall 211 and the communicating pipe 212, and a pipe opening of the communicating pipe 212 is plugged through a pipe sealing part 214 arranged on the cover. In this embodiment, the shape of the cover 213 is the same as that of the upper cavity wall 211 and forms an overturning cover structure of the reactor cavity, one side of two opposite sides of the cover 213 is connected with a joint flange 215, and a fixed buckle 216 is arranged between the other side of the cover and the joint flange 215. The joint of the cover 213 and the joint flange 215 is provided with a positioning crease 217, and the positioning crease 217 is used for making the cover 213 bent from the same position when the cover 213 is overturned. When the cover 213 loosely covers the upper cavity wall 211 of the reactor and the communicating pipe 212, the aerated culture of the reactor can be maintained; when the cover 213 tightly covers the upper cavity will 211 of the reactor and the communicating pipe 212, the other side of the cover 213 is tightly buckled with a buckle 216 of the joint flange. The cover 213 and the upper cavity wall 211 are in adhesion clearance closing with each other, and the pipe sealing part 214 plugs the pipe opening of the communicating pipe 212, so that anaerobic culture or storage of reactants after culture can be performed so as to prevent liquid evaporation.

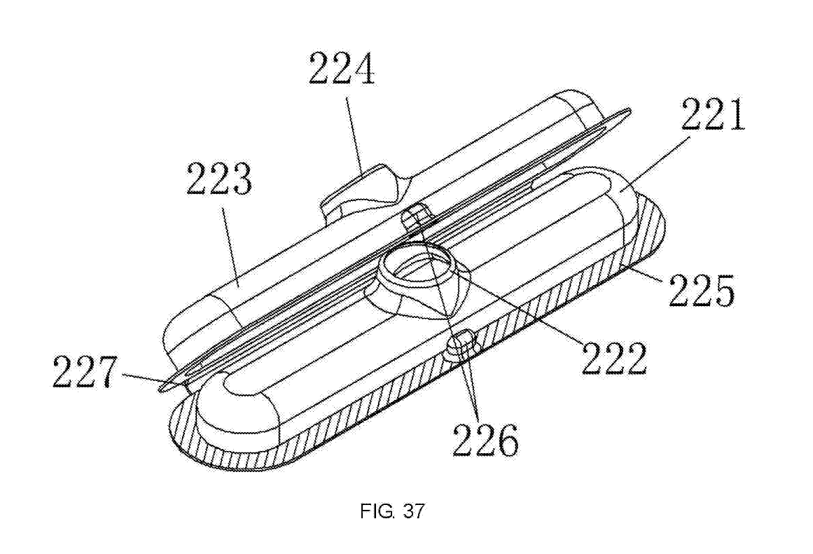

Embodiment 22 of the disposable bioreactor is as shown in FIG. 37. This embodiment is different from Embodiment 5 in that in this embodiment, the reactor is further provided with a cover 223, and the cover 223 is in fact an extension structure of a pipe cap of a communicating pipe 222, is used for entirely covering an upper cavity wall 221 and the communicating pipe 222 and plugs a pipe opening of the communicating pipe 222 through a pipe sealing part 224 arranged on the cover 223. In this embodiment, the shape of the cover 223 is the same as that of the upper cavity wall 221 and forms an overturning cover structure of the reactor cavity, one side of two opposite sides of the cover 223 is connected with a joint flange 225, and a fixed buckle 226 is arranged between the other side of the cover 223 and the joint flange 225. The joint of the cover 223 and the joint flange 225 is provided with a positioning crease 227, and the positioning crease 227 is used for making the cover 223 bent from the same position when the cover 223 is overturned. When the cover 223 loosely covers the upper cavity wall 221 of the reactor and the communicating pipe 222, the aerated culture of the reactor can be maintained; when the cover 223 tightly covers the upper cavity wall 221 of the reactor and the communicating pipe 222, the other side of the cover 223 is tightly buckled with a buckle 226 of the joint flange. The cover 223 and the upper cavity wall 221 are in adhesion clearance closing with each other, and the pipe sealing part 224 plugs the pipe opening of the communicating pipe 222, so that anaerobic culture or storage of reactants after culture can be performed so as to prevent liquid evaporation.

In each aforementioned embodiment, the disposable bioreactor undergoes irradiation sterilization in advance and is disposable in real time, and the trouble in cleaning and sterilization and the risk of cross contamination are avoided.

In other embodiments of the disposable bioreactor, the reactor cavity can further be formed by more than three cavity walls through fusion connection or adhesion connection. When there are more than four cavity walls, each cavity wall is not necessarily a curved surface cavity wall, and a hollow arm part of the reactor cavity can be formed by splicing a plurality of cavity walls. In addition, in other embodiments, the upper cavity wall and the lower cavity wall of the reactor can further be soft film cavity walls. Due to the existence of the hollow arm part, even if the upper cavity wall and the lower cavity wall are made of soft films, air blowing is not needed in the using process, and a three-dimensional space in the reactor cavity can be maintained only by means of pulling fixation and the like. Moreover, one of the left cavity wall and the right cavity wall can be a fixed-shaped supporting cavity wall and the other is a soft film cavity wall, or the left cavity wall and the right cavity wall are both soft film cavity walls. The reactor cavity can be made of a medical latex film, a silica gel film, etc. In Embodiments 3 and 4, the upper cavity wall can further be a plane soft film cavity wall; under the conditions, due to the supporting effects of the joint flange and the lower cavity wall, the upper cavity wall can automatically maintain an unfolded state and does not fall, and the three-dimensional space in the reactor cavity can be maintained with no need of air blowing and pulling.

We claim:

1. A disposable bioreactor, comprising a reactor cavity and a communicating pipe located at the top of the reactor cavity, wherein the reactor cavity is formed by more than two cavity walls through fusion connection or adhesion connection and is provided with an intersection part and at least one hollow arm part which is communicated with the communicating pipe through the intersection part in an intersection manner, and the tail end of the hollow arm part is sealed.

2. The disposable bioreactor according to claim 1, wherein the hollow arm part is arranged horizontally.

3. The disposable bioreactor according to claim 1, wherein the reactor cavity is provided with one hollow arm part and the communicating pipe is located at the top of the reactor cavity and forms an L-shaped structure together with the hollow arm part.

4. The disposable bioreactor according to claim 1, wherein the reactor cavity is provided with two hollow arm parts, and the communicating pipe is located at the top of the reactor cavity and forms an inversely T-shaped structure together with the hollow arm parts.

5. The disposable bioreactor according to claim 1, wherein the reactor cavity is provided with two cavity walls which are arranged up and down; one cavity wall located on the upper portion is an upper cavity wall and the other cavity wall is a lower cavity wall; and at least one of the upper cavity wall and the lower cavity wall is a curved surface cavity wall.

6. The disposable bioreactor according to claim 5, wherein one of the upper cavity wall and the lower cavity wall is a fixed-shaped supporting cavity wall capable of maintaining an inherent shape, and the other is a soft film cavity wall.

7. The disposable bioreactor according to claim 6, wherein the fixed-shaped supporting cavity wall is formed by plastic raw materials through injection moulding or formed by plastic sheets through plastics sucking moulding or blow molding.

8. The disposable bioreactor according to claim 6, wherein the soft film cavity wall is formed by a plane plastic film or formed by a deformable soft film bubble formed by the plane plastic film through plastics sucking moulding.

9. The disposable bioreactor according to claim 6, wherein the upper cavity wall and the lower cavity wall are curved surface cavity walls; the fixed-shaped supporting cavity wall is an overlapped type cavity wall capable of being overlapped in a sleeved manner, and the soft film cavity wall can be folded into the fixed-shaped supporting cavity wall so that the reactor cavity can be overlapped in a sleeved manner.

10. The disposable bioreactor according to claim 5, wherein one of the upper cavity wall and the lower cavity wall is a curved surface cavity wall and the other is a plane cavity wall.

11. The disposable bioreactor according to claim 5, wherein the communicating pipe and the upper cavity wall are of an integrated structure.

12. The disposable bioreactor according to claim 11, wherein an outer port of the communicating pipe is formed by punching after plastics sucking moulding, and a reinforcing ring which surrounds the outer port and remains after the punching is positioned at the position of the outer port of the communicating pipe.

13. The disposable bioreactor according to claim 5, wherein the lower cavity wall is the curved surface cavity wall; the bioreactor further comprises a supporting fixing base which is provided with a yielding groove used for yielding to the lower cavity wall, and a fixing structure for fixing or pulling the reactor cavity is arranged on the periphery of a notch of the yielding groove.

14. The disposable bioreactor according to claim 5, wherein the joint of the upper cavity wall and the lower cavity wall is provided with a joint flange which extends towards the periphery and is used for supporting, pulling and fixing the reactor cavity.

15. The disposable bioreactor according to claim 14, wherein the reactor further comprises a cover for covering the upper cavity wall of the reactor cavity, and the cover is provided with a pipe sealing part for plugging a pipe opening of the communicating pipe.

16. The disposable bioreactor according to claim 14, wherein the cover forms an overturning cover structure of the reactor cavity; one of two opposite sides of the cover is connected with the joint flange, and a fixed buckle is arranged between the other side of the cover and the joint flange.

17. The disposable bioreactor according to claim 1, wherein the cavity wall of the reactor cavity is equipped with a pipe communicated with the reactor inner cavity so that liquid and gas can go in and out and dissolved oxygen, carbon dioxide and PH sensor electrodes can pass, and the pipe communicated with the reactor inner cavity and the connected cavity wall are of an integrally formed structure.

18. The disposable bioreactor according to claim 1, wherein the reactor cavity is formed by two cavity walls through fusion connection or adhesion connection; one of the two cavity walls is a front cavity wall and the other is a rear cavity wall; the matching joint of the front cavity wall and the rear cavity wall is provided with a joint flange, and a hollow arm part of the reactor cavity is formed by corresponding parts of the front cavity wall and the rear cavity wall.
